(19) United States
(12) Patent Application Publication
(10) Pub. No.: US 2006/0112961 A1
(43) Pub. Date: Jun. 1, 2006

(54) Pediatric mask, especially suited for premature infants

(76) Inventor: Hany Aly, Alexandria, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/998,016

(22) Filed: Nov. 29, 2004

(51) Int. Cl. A62B 7/10; A61M 15/08

(52) U.S. Cl. 128/206.11; 128/207.18

(57) ABSTRACT

The inventive masks and medical gas-delivery systems make possible delivery of medical gases (such as oxygen) to pediatric patients with faces too small to be comfortably served by conventional masks and systems sized for adults. The inventive masks minimize the surface area of the baby's face being covered and also balance tubing and related parts contacting the baby. In addition, it allows efficient delivery of required pressure, while minimizing any leak of delivered oxygen or gas outside the breathing system.

### BACKGROUND OF THE INVENTION

1. Field of the Invention

The present invention generally relates to pediatric masks, especially suited for premature infants.

2. Background Description

Oxygen face masks for medical use have been known conventionally, such as U.S. Pat. No. 3,827,433 to Shannon, “Respiratory device and procedure” (issued Aug. 6, 1974); U.S. Pat. No. 4,641,647 to Behan, “Device for securing respiratory appliance during respiratory therapy” (issued Feb. 10, 1987); U.S. Pat. No. 4,799,477 to Lewis, “Rebreathing Mask” (issued Jan. 24, 1989); U.S. Pat. No. 4,832,017 to Schnoor, “Breathing Mask” (issued May 23, 1989); U.S. Pat. No. 4,848,334 to Bellm, “Mask” (issued Jul. 18, 1989); U.S. Pat. No. 5,400,781 to Davenport, “CO₂ gas sampling mask having a beveled sampling tube extending into the mask” (issued Mar. 28, 1995); U.S. Pat. No. 5,758,642 to Choi, “Improved gas delivery mask” (issued Jun. 2, 1998); U.S. Pat. No. 5,704,073 to Sword et al., “Quick donning goggles for use with breathing mask” (issued Jan. 6, 1998); U.S. Pat. No. 6,422,238 to Lithgow, “Headgear” (issued Jul. 23, 2002); U.S. Pat. No. 6,581,594 to Drew et al., “Respiratory mask having gas washout vent and gas washout vent for respiratory mask” (issued Jun. 24, 2003).

For patients suffering from obstructive sleep apnea and other breathing disorders, administering non-invasive ventilation treatment (such as, e.g., continuous positive airway pressure (CPAP)) has been known. See, e.g., U.S. Pat. No. 5,560,354 to Berthon-Jones et al., “Facial masks for assisted respiration or CPAP” (issued Oct. 1, 1996); U.S. Pat. No. 5,746,201 to Kidd, “CPAP nose mask” (issued May 5, 1998). For adult patients, CPAP has been delivered by a positive airway pressure generator into a mask worn by the patient while sleeping.

CPAP also has been applied for pediatric patients. See, e.g., Stenzler, U.S. patent application No. 2002/0078958 A1, published Jun. 27, 2002, for “Infant CPAP system with airway pressure control,” disclosing a device for delivering CPAP to an infant.

However, it must be kept in mind that pediatric patients are unable to communicate, understand or adjust physically as a non-pediatric patient typically can, and that masking a face of a pediatric patient may involve more than simply a question of scaling an adult system to a pediatric size. Bohn et al., U.S. patent application No. 2003/0145859 A1 (published Aug. 7, 2003), titled “Pediatric Ventilation Mask and Headgear System,” discloses certain masks that form animal characters, such as an elephant. The masks covering the least of a child's face include straps across each side of the child's face and around the back of his head, as well as third strap across the forehead connecting the nosepiece to the cap (with the ornamental ears). However, Bohn et al.'s animal-character masks assume a patient able to comprehend the fun aspect of the mask.

CPAP for the smallest patients, especially premature newborns, remains in need of improvement. Improved methods of ventilating pediatric patients, especially premature infants, remain desirable.

### SUMMARY OF THE INVENTION

It is therefore an object of the present invention to provide new methods of ventilating patients, especially pediatric patients (such as, e.g., a pediatric patient having a head circumference of about 20 inches or less; a premature infant having a head circumference of about 15 inches or less; etc.), and most especially premature newborn infants. It is a further object of the present invention to provide new methods, systems and products addressing the shortcomings of the conventional technology and methods discussed above in the Background.

In one preferred embodiment, the invention provides a pediatric gas-delivery system, comprising: a gas source of a gas to be supplied to a pediatric patient (such as, e.g., a pediatric patient having a head circumference of about 20 inches or less; a premature infant having a head circumference of about 15 inches or less; etc.); at least one supply channel through which may travel gas from the gas source to the patient's nose and/or mouth; and at least one exit channel receiving the patient's expired air, with the channels arranged in a balanced structure, the balanced structure including or joining with a pediatric-sized mask covering at least one of the patient's nose and mouth (such as, e.g., a pediatric-sized mask having in the mask at least one pediatric-sized nose-hole and/or at least one pediatric-sized mouth-hole). A preferred embodiment provides a mask having exactly one hole, the one hole being a nose-hole, a mouth-hole, or a nose-and-mouth-hole.

In another preferred embodiment, the invention provides a gas-delivery system, comprising: at least two tube sections, the tube sections being disposed wherein gas from a gas source travels through at least one of the tube sections towards at least one of a patient's nose and mouth, the tube sections being structurally disposed with no tube section being permitted to enter any interior of the patient's nose and mouth.

In a further preferred embodiment, the invention provides a pediatric mask, comprising: a mask sized to fully cover at least one of a nose and a mouth of a pediatric patient having a head circumference less than about 20 inches; the mask, when fitted onto to the pediatric patient, having no opening other than a hollow structure that extends away from the mask in a direction away from the patient and after extending branches into a first connector opening and a second connector opening, the first and second connector openings balancing each other, and each connector opening capable of receiving insertion of a tube for delivery of a medical gas or transit of expired air from the patient.

In the inventive systems and masks, some optional perfecting details are as follows. Preferably, the mask extends in a direction away from the mouth and/or nose the mask into a bridge-like channel which branches into a left opening and a right opening, each of which opening detachably receives a respective inspiratory tube or expiratory tube. Preferably, the mask covers only as much of an area around the nose and/or mouth as needed to fit snugly, and the face is largely uncovered.

Preferably, in the inventive systems and masks, the gas used (such as oxygen, etc.) travels into both a pediatric patient's nose and mouth. A preferred rate of delivery of the gas from the gas source is a rate in a range of about 5 to 20 L/minute. Preferably, during gas delivery, a snug seal around the nose and/or mouth being masked is provided with regard to the mask through which the gas travels. Preferably, the mask covers only as much of an area around the nose and/or mouth as needed to fit snugly, and the face is largely uncovered.

In the inventive gas-delivery systems and masks, where tube sections are used, preferably the tube sections are connected to a mask, directly or via a connection structure. The tube sections may be, for example, a respective left tube and a respective right tube, from a perspective of the patient. Two tube sections may join at a balanced branch point in a preferred configuration. When a balanced branch point is used, the balanced branch point may, for example, connect to the mask indirectly (such as via a hollow channel), or directly. When a balanced branch point connect to a mask via a hollow channel, preferably the mask, the hollow channel and the balanced branch point are formed as a unitary piece (such as, e.g., a unitary piece having two respective openings for respectively receiving the two tube sections, each tube section being insertible into, and detachable from, the unitary piece). Where two tube sections are used, preferably one tube section mainly provides gas that the pediatric patient inspires, and the other tube section mainly receives gas expired by the pediatric patient.

In the inventive systems and masks, preferably the mask is fastened to the patient with minimal bother to the patient, such as by a strap circling the pediatric patient's head. Preferably a head cap is included.

In a particularly preferred embodiment of the invention, a mask is sized to a small-sized patient, such as, e.g., a pediatric patient having a head circumference of about 20 inches or less, a premature infant having a head circumference of about 15 inches or less, etc.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a perspective view looking down on a pediatric patient, lying on his back, having oxygen administered according to the present invention.

FIG. 2 is a cross-sectional end-view of an inventive maskpiece, as in FIG. 1, for the end placed onto the patient's nose and mouth.

FIG. 3 is a top view of an inventive mask structure as used in FIG. 1.

FIG. 4 is a cross-sectional view along line 4-4 in FIG. 3.

It will be appreciated that the figures herein are not to scale.

### DETAILED DESCRIPTION OF A PREFERRED EMBODIMENT OF THE INVENTION

A preferred embodiment of the invention is described with reference to the drawings.

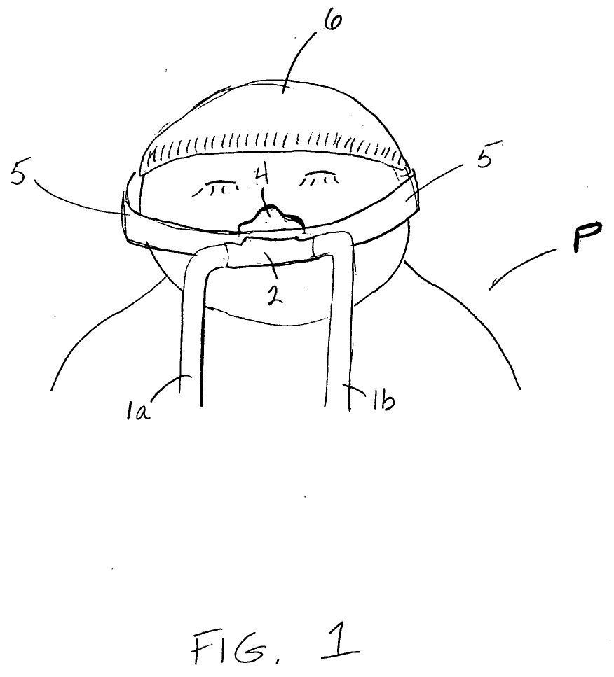

Referring to FIG. 1, there is shown a system for delivering a gas (e.g., preferably, a gas that comprises oxygen, most preferably, pure oxygen) to a patient P (preferably a pediatric patient, most preferably a premature newborn infant) via the nose and/or the mouth. The oxygen and/or gas mixture travels from an oxygen source through either tubing 1a or 1b, depending on which of tubing 1a or 1b is connected to the oxygen source. Carbon dioxide expired by the patient travels away from the patient throw tubing 1b or 1a, depending on which is not connected to the oxygen source. That is, expired air travels through the tubing which is not connected to the oxygen source.

In FIG. 1, tubing 1a, 1b is detachably connected to and received by a hollow T-shaped structure 2.

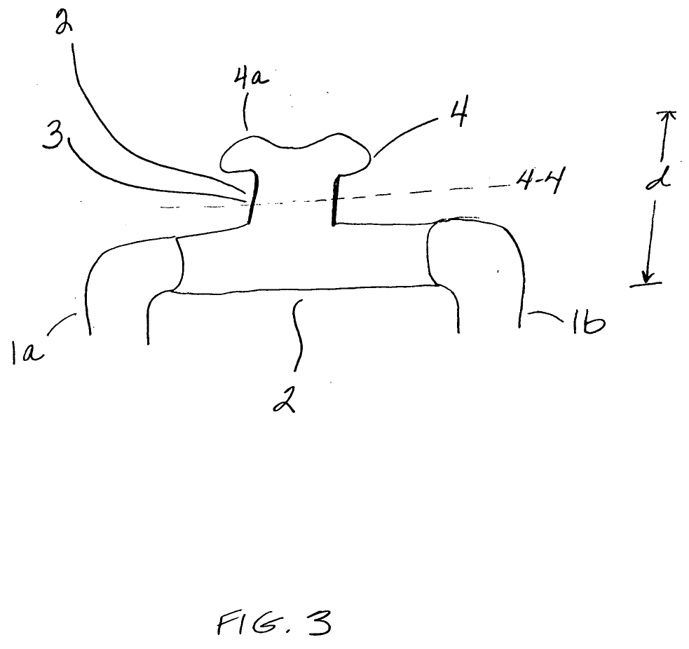

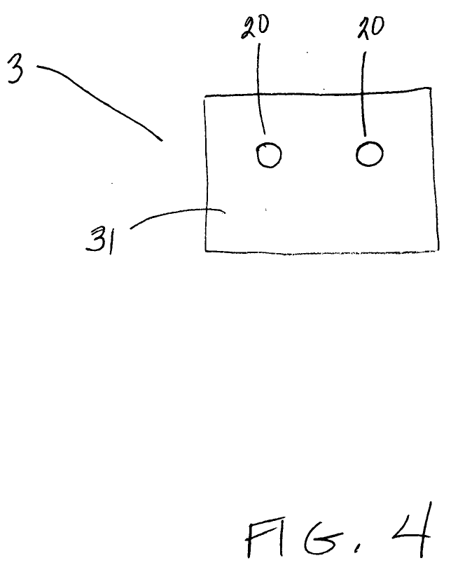

Referring to FIG. 3, the base 3 (not shown in FIG. 1) of the “T” shape structure 2 includes hollow space (such as two holes or tubes 20, see FIG. 4) for permitting incoming gas mixture or oxygen and outgoing expired air to pass. A solid part 31 of the base 3 is solid with the holes or tubes 20 defining a hollow portion, but the base 3 is not required to be solid throughout and may open into a hollow section. The length and diameter of the holes or tubes 20 may be selected for controlling passage of the gas therethrough. An example of a suitable length of the holes 20 is 1 cm. An example of a suitable diameter for the holes 20 is 0.5 cm.

Referring to FIGS. 1 and 3, the “T” shape structure 2 terminates at the mask facepiece 4, with the mask facepiece 4 being open for placing over, and covering, the mouth and/or nose of the patient P. An example of a material from which to construct a mask according to the present invention is, e.g., a soft flexible and/or adhesive rubber such as, e.g., poly vinyl chloride (PVC), silicone, douderm, etc. The inventive mask is constructed to include a hollow mask facepiece 4 sized for receiving a patient's nose and/or mouth, most preferably, sized for receiving a premature newborn infant's nose and/or mouth.

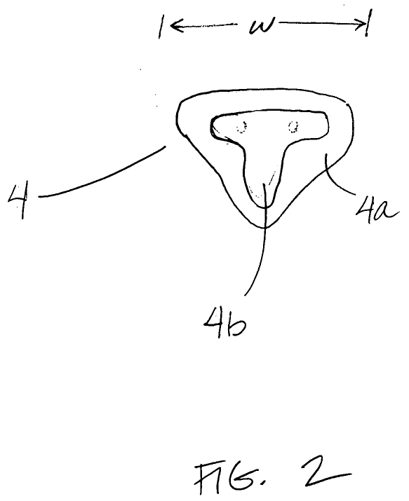

As shown in FIG. 2, bottom 4a of facepiece 4 contacts and/or sticks to the patient's face. The hollow cut-out 4b of the facepiece 4 receives the nose of the patient.

Returning to FIG. 1, a strap 5 has a rectangular cut-out section (not shown in FIG. 1) which receives the base 3 of the T-shaped structure. The T-shaped structure 2, being of soft flexible rubber (such as PVC, etc.), can be inserted and removed from the cut-out section of the strap 5. The strap 5 is no thicker, heavier or wider than needed for support, and is as thin and light as possible for comfort of the patient P. The respective right and left parts of the strap 5 pass behind the patient's head and fasten, by Velcro, to a matching Velcro section on the back of the patient's head cap 6.

In an exemplary pediatric mask having a hollow structure, a maximal width w (referring to FIG. 2) for the hollow structure (with the width measured in a right/left direction with respect to the patient's face) that may be used is a dimension appropriate for an infant, such as, e.g., a maximum width of a bit more than 1 inch, such as, e.g., 1⅛ inches, however, the width in an inventive mask is not limited thereto. It will be appreciated that preferably a maximal width of mask may be used at the point where the mask covers the infant's nose, and that the width of the mask may taper to a smaller width at a point just below where the mask covers the baby's mouth.

A pediatric mask may be made in which a depth dimension d (see FIG. 3) from where the mask is fitted onto the pediatric patient to a point of the hollow structure extending straight away from the patient and farthest from the patient is an amount for balancing the related tubing, such as, preferably, when ⅜ oxygen-delivery tubing is used, a depth dimension for the mask of about 1⅛ inches; however, the depth dimension is not limited thereto. For example, if tubing of smaller diameter than ⅜ inch diameter tubing can be used for connecting to the mask, it may be possible to reduce the depth dimension of the mask under 1⅛ inch while still achieving a balanced structure.

For the diameter of the connector openings (where tubing 1a meets T-shaped structure 2 in FIG. 3) in an exemplary pediatric mask, a preferred diameter (outer wall to outer wall) is about ⅜ inches. However, the diameter is not limited thereto, and, for example, the diameter (outer wall to outer wall) may be such as is used in oxygen or other gas-delivery systems. In general, connector openings are desired to be as small in diameter as practicable for delivery of oxygen (or other gas(es)), so as not to unnecessarily increase other dimensions of the pediatric mask structure.

Thus, the invention provides a minimally-dimensioned, minimally invasive, minimally disturbing mask structure designed to keep as much of the pediatric patient's face uncovered as feasible while accomplishing, and without compromising, the medical objective of delivery of oxygen or the other gas(es) as needed.

The present invention advantageously minimizes the surface area of the pediatric patient's face that is covered by one or more parts of a system that is delivering oxygen or another medical gas mixture to a pediatric patient.

It will be appreciated that the inventive mask and system as show in FIGS. 1-4 may be used for delivery of continuous positive airway pressure (CPAP) to premature newborn patients (and other larger patients) suffering from respiratory distress or apnea.

### COMPARATIVE EXAMPLE 1

A conventional device for delivery of CPAP is by putting a mask on a patient face, where the mask is attached to the face on one side, while the other side is attached to the flow source. The expiratory air exits either through some holes in the mask, or through an expiratory port fixed to the flow source. Though easy to apply to a patient, several problems are encountered with this device. First, it is difficult to achieve a good seal of the mask to the face, mostly because of the holes on the mask, or due to the attachment of the large flow tube to the middle of the mask. Therefore, any swinging movement of that flow tube will break the seal of the relatively small mask away from the face of the patient. Second, to overcome the afore-mentioned problem, some clinicians tie the mask tightly on the nose. However, such tying causes significant trauma to the nose. Third, the interface of the inspiratory flow and the expiratory air inside the mask and through the tubing is not optimum and can possibly interfere with easy expiration.

### COMPARATIVE EXAMPLE 2

A second conventional device for CPAP delivery is by using two nasal prongs that fit inside the nasal opening of the patient. This device can have better fit and seal to the patient nose compared to the device of Comparative Example 1. However, the major drawback with the device of this Comparative Example 2 is that, with its application to the nose, significant trauma is caused to the fragile nasal mucosa and to the nasal septum. Damage and nasal bleeding often occurs when using nasal pronged CPAP devices. Many ICU units have abandoned the use of CPAP completely because of this problem.

### EXAMPLE 1

### (HI-MASK)

A pediatric mask structure has been constructed from poly vinyl chloride (PVC), with the following dimensions:
Maximum width, at part of mask structure near patient's face (left to right dimension): ˜1⅛ inches
Depth, from where the mask is fitted onto the patient to a point of the hollow structure extending straight away from the patient: ˜1⅛ inches
Diameter (outer wall to outer wall) of connector openings: ˜⅜ inches
Narrowest width (left to right) of the hollow structure ˜⅝ inches
   A pediatric maskpiece dimensioned according to this Example 1 was made of a soft, pliable plastic. The pediatric maskpiece was used in conjunction with a strap having a cutout into which was inserted the maskpiece, at the narrowest width of the mask structure. The strap (attached to a cloth headcap) were used to hold the mask on an infant positioned on his back.

A mask according to the invention may be constructed as one piece, such as, most preferably, a one-piece mask made of a soft flexible rubbery material, such as, e.g., poly vinyl chloride (PVC), silicone, etc. For forming the shape of the mask, the mask material (such as PVC, silicone, etc.) may be injected into a mold (such as a mold composed of two plates, which when facing each other have therebetween a cavity that defines the shape of the mask).

### EXAMPLE 2

The pediatric mask according to Example 1 was used by the present inventor on at least one pediatric patient who was a baby. The pediatric patient was sleeping, back down, face up. Through one length of medical tubing attached to the mask, oxygen was delivered via the mask to the sleeping baby, through the nose. Expired air from the baby exited from the mask via a second length of medical tubing attached to the mask.

It was concluded, by using a mask according to invention in the delivery of oxygen to pediatric patients, that the inventive mask and an oxygen-delivery system according to the present invention facilitate delivery of oxygen to pediatric patients with faces too small to be comfortably served by conventional masks and systems sized for adults. The inventive mask minimizes the surface area of the baby's face being covered and also balances tubing and related parts coming into contact with the baby's face, thus minimizing distress to the patient who is a baby.

The device and system according to inventive Examples 1 and 2 overcomes all the shortcomings mentioned for Comparative Examples 1 and 2. The inventive mask has no expiratory opening that may interfere with the tight seal. The inventive mask has an inspiratory tube to run on one side of the face and an expiratory tube on the other side of the face, allowing efficient ventilation. There is a bridge between the two tubes that allows the flow to pass through and facilitates expiration by the patient. The position of the two tubes on the sides of the face facilitates balance of the mask on the face and allows the seal on the nose without the need for tight ties or trauma to the face or nose.

The actual use of the inventive high CPAP mask device showed its efficiency when applied to patients for several weeks, without any noticeable trauma or marks on the face or nose. Nursing staff was able to easily apply the inventive mask without need for frequent position adjustment as was needed with conventional devices (Comparative Examples 1, 2). Strikingly, efficient ventilation using the invention is seen as evidenced by the maintenance of normal blood gases during its use. Accumulation of carbon dioxide in particular has been a concern with the use of conventional CPAP devices. By contrast, carbon dioxide levels are relatively normal in the blood gases of patients using the inventive mask and system.

While the oxygen and medical-gas delivery systems of the present invention have been discussed above with particular regard to premature infants and newborns, it will be appreciated that the invention also may be extended to older and larger patients, especially those who lack usual adult reasoning and understanding to be able to tolerate masking on their faces. That is, a mask system according to the present invention optionally may be scaled-up.

Advantageously, in preferred embodiments, the inventive masks and medical gas-delivery systems make possible delivery of medical gases (such as oxygen) to pediatric patients with faces too small to be comfortably served by conventional masks and systems sized for adults. Inventive masks may be provided that minimize the surface area of the baby's face being covered and also balance tubing and related parts contacting the baby. In addition, the invention advantageously allows efficient delivery of required pressure, while minimizing any leak of delivered oxygen or gas outside the breathing system.

While the invention has been described in terms of its preferred embodiment, those skilled in the art will recognize that the invention can be practiced with modification within the spirit and scope of the appended claims.

Having thus described our invention, what I claim as new and desire to secure by Letters Patent is as follows:

1. A pediatric gas-delivery system, comprising:
a gas source of a gas to be supplied to a pediatric patient;
at least one supply channel through which may travel gas from the gas source to the patient's nose and/or mouth; and
at least one exit channel receiving the patient's expired air, with the channels arranged in a balanced structure, the balanced structure including or joining with a pediatric-sized mask covering at least one of the patient's nose and mouth.

2. The pediatric system of claim 1, wherein the mask extends in a direction away from the mouth and/or nose the mask into a bridge-like channel which branches into a left opening and a right opening, each of which opening detachably receives a respective inspiratory tube or expiratory tube.

3. The pediatric delivery system of claim 1, wherein the mask covers only as much of an area around the nose and/or mouth as needed to fit snugly, and the face is largely uncovered.

4. The pediatric delivery system of claim 1, wherein the gas travels into both a pediatric patient's nose and mouth.

5. The pediatric delivery system of claim 1, wherein the mask is sized to snugly fit over the pediatric nose and/or mouth being covered.

6. The pediatric delivery system of claim 1, wherein, during gas delivery, a snug seal around the nose and/or mouth being masked is provided.

7. A gas-delivery system, comprising:
at least two tube sections,
the tube sections being disposed wherein gas from a gas source travels through at least one of the tube sections towards at least one of a patient's nose and mouth,
the tube sections being structurally disposed with no tube section being permitted to enter any interior of the patient's nose and mouth.

8. The gas-delivery system of claim 7, wherein the tube sections are connected to a mask, directly or via a connection structure.

9. The gas-delivery system of claim 7, wherein the patient is a pediatric patient having a head circumference of about 20 inches or less.

10. The gas-delivery system of claim 9, wherein the patient is a premature infant having a head circumference of about 15 inches or less.

11. The pediatric delivery system of claim 9, including a pediatric-sized mask having in the mask at least one selected from the group consisting of at least one pediatric-sized nose-hole and at least one pediatric-sized mouth-hole.

12. The pediatric delivery system of claim 9, wherein the tube sections are a respective left tube and a respective right tube, from a perspective of the patient.

13. The pediatric delivery system of claim 11, including two tube sections that join at a balanced branch point.

14. The pediatric delivery system of claim 13, wherein the balanced branch point connects to the mask indirectly via a hollow channel, or directly.

15. The pediatric delivery system of claim 14, wherein the balanced branch point connects to the mask via a hollow channel, with the mask, the hollow channel and the balanced branch point being formed as a unitary piece.

16. The pediatric delivery system of claim 15, wherein the unitary piece has two respective openings for respectively receiving the two tube sections, each tube section being insertible into, and detachable from, the unitary piece.

17. The pediatric delivery system of claim 9, wherein one tube section mainly provides gas that the pediatric patient inspires, and the other tube section mainly receives gas expired by the pediatric patient.

18. The pediatric delivery system of claim 11, including a strap circling the pediatric patient's head.

19. A pediatric mask, comprising:
a mask sized to fully cover at least one of a nose and a mouth of a pediatric patient having a head circumference less than about 20 inches;
the mask, when fitted onto to the pediatric patient, having no opening other than a hollow structure that extends away from the mask in a direction away from the patient and after extending branches into a first connector opening and a second connector opening, the first and second connector openings balancing each other, and each connector opening capable of receiving insertion of a tube for delivery of a medical gas or transit of expired air from the patient.

20. The pediatric mask of claim 19, sized to a pediatric patient having a head circumference of about 20 inches or less.
